(19) United States
(12) Patent Application Publication
(10) Pub. No.: US 2004/0137534 A1
(43) Pub. Date: Jul. 15, 2004

(54) Methods for detecting deantigenized T cell epitopes and uses thereof

(76) Inventor: Subhashis Banerjee, Shrewsbury, MA (US)

Correspondence Address: ABBOTT BIORESEARCH, 100 RESEARCH DRIVE, WORCESTER, MA, 01605-4314, US

(21) Appl. No.: 10/625,804

(22) Filed: Jul. 23, 2003

Related U.S. Application Data
(60) Provisional application No. 60/397,758, filed on Jul. 23, 2002.

(51) Int. Cl. G01N 33/53; G01N 33/567

(52) U.S. Cl. 435/7.2

(57) ABSTRACT

The present invention encompasses a novel approach to develop modified proteins that induce a reduced (or no) immune response (compared to an unmodified protein) in an animal, wherein one or more T cell epitope sequences present in the unmodified protein is/are replaced with one or more “deantigenized” T cell epitope sequences, which exhibit reduced or no binding to a MHC molecule (compared to the T cell epitope); thereby hindering the cellular process of immune response. The present invention provides a novel method for detecting deantigenized T cell epitopes and further reducing or eliminating immunogenicity of an otherwise immunogenic protein by substituting one or more deantigenized T cell epitope sequences for one or more T cell epitope sequences of the parent immunogenic polypeptide.

### FIELD OF THE INVENTION

The present invention is in the fields of cellular and immunobiology. The present invention is directed to novel methods for detecting deantigenized T cell epitopes, nonimmunogenic or reduced immunogenic therapeutic polypeptides derived therefrom, and their uses.

### BACKGROUND OF THE INVENTION

The development of protein molecules as therapeutics continues to offer increasing benefit and hope to the medical community. Use of therapeutic proteins extends beyond conventional use of proteins as vaccines, whereby the protein functions to induce an immune response in an animal. Other therapeutic or prophylactic applications of proteins and polypeptides may include but are not limited to proteins that: replace, substitute, or augment endogenous proteins, e.g. hormones; exhibit enzymatic activity; act as adjuvants (including proteins capable of converting inactive drugs to active drugs in an animal); function as carriers for other molecules (e.g., proteins capable of extending the biological half-life of a molecule, including endogenous molecules, in an animal); and are capable of binding to molecules within or administered to an animal to alter the bioactivity, biodistribution and/or bioavailability of the bound molecules.

Unlike vaccines, the benefits of these other therapeutic proteins are greatly reduced if they induce an immune response in the animal (including human) being treated. This is particularly true in cases where multiple dose applications of the therapeutic protein are required to achieve or to maintain its beneficial effect(s). Eliciting an undesirable immune response may result in i) the neutralization and clearance of the therapeutic protein, thus reducing or preventing its beneficial effect in the treated animal, and, of more serious import, ii) development of an allergic response in the treated animal to the therapeutic protein, putting the animal at risk of anaphylactic shock upon further exposure to the protein. A well-known example of the undesirable induction of an immune response to a therapeutic protein is the human immune response upon exposure to a mouse-derived protein (e.g., murine antibodies), whereby the human subject produces Human Anti-Murine Antibodies; known as the HAMA response.

In contradistinction to the discovery and development of protein vaccines to induce an immune response in an animal, development of these non-vaccine therapeutic proteins should seek to minimize (e.g., reduce or eliminate) any immune response in an animal to the protein.

Several techniques specifically addressing the HAMA response are known in the art. One approach has been to “humanize” the therapeutic protein (typically a murine-derived monoclonal antibody) by the introduction/replacement of polypeptide regions (e.g., in the mouse sequence) with amino acid sequences identical to those present in the sequence of a human protein analogue or homologue, thus rendering the re-modeled protein non-immunogenic (e.g., see Adair et al., 1991; Law et al., 1991; Queen, 1989; and Winter, 1989). This approach has met with limited success, however (e.g., specific short peptide sequences, “T cell epitopes”, which trigger T cell activation when presented by the Major Histocompatibility Complex, or “MHC”, may persist in the molecule).

It has been discovered that even proteins of human origin (including fully human proteins such as human insulin) are capable of inducing an immune response in a human subject, whereby the human subject produces Human Anti-Human Antibodies; known as the HAHA response (Ritter et al., 2001).

Another approach to reduce the immunogenicity of a protein in an animal is to identify T cell epitope(s) of the protein and render it (them) non-immunogenic by amino acid modification (e.g., Carr, 1998); a process referred to herein as the “deantigenizing” of T cell epitopes.

Here, the T cell epitopes are identified by computational or physical methods establishing MHC binding in silico or on the surface of cells. The amino acid sequence of identified T cell epitopes are modified and the modified T cell epitopes are tested using a MHC expressing cell-based assay. Carr et al. (2000) further disclose a method for the elimination of “self” epitopes, which may give rise to immune reactions, by recombinant DNA technology.

Warmerdam et al. (2001) provide a method for reducing the immunogenicity of a protein, by designing a series of overlapping test peptides, each having an amino acid sequence that corresponds with part of the amino acid sequence of the protein of which the immunogenicity is to be reduced. The overlapping test peptides are tested for the ability to activate antigen-specific receptors on T cells (T cell receptors), thereby identifying T cell epitopes. The amino acid sequence of positively identified T cell epitopes are then modified and retested. This process is repeated until a modified T cell epitope with reduced ability to activate T cell receptors is identified. The amino acid sequence of the reduced immunogenic T cell epitope is then substituted into the full protein sequence.

Current approaches (including those above) to reduce immunogenicity of proteins present several problems and obstacles for the practitioner, however.

Computer modeling techniques are currently employed to predict T cell epitope binding characteristics to MHC molecules in silico. These techniques include amino acid sequence comparison analysis to known MHC binding motifs, algorithms to predict MHC binding, or “peptide threading” in silico using models of known X-ray structures of MHC molecules (Altuvia et al., 1995). These methods are not wholly predictive of actual peptide binding, however, due to variabilities in the manner in which peptides may actually bind to the same or similar MHC molecules in vivo (e.g., as a result of subtle configurational changes in either or both the T cell epitope or MHC molecule on binding to each other). False positive or negative results may occur as a result. Such computer predictions must still be validated by cellular assays.

Known cellular assays used to characterize peptide immunogenicity include cell surface binding assays, which depend on peptide (T cell epitope) binding to cell lines, such as B lymphocyte lines, expressing unique MHC molecules. These assays involve either direct binding or inhibition of binding of known peptides to the MHC molecules on the cell surface. Alternative assays include T cell proliferation assays known in the art (e.g., Estell and Harding, 1999; Stickler et al., 2000). T-lymphocyte proliferation requires both appropriate peptide presentation by MHC molecules and T cell receptor recognition. Consequently, T cell assays alone do not discriminate between MHC binding and T cell activation effects. All of these assays involve either direct binding or inhibition of binding of known peptides to the cell surface.

As appreciated by practitioners in the art, these cellular assays suffer several drawbacks; not the least of which are low sensitivity, and poor reproducibility from lab to lab and from reagent to reagent. In addition, it is difficult, yet critical, to demonstrate that peptide binding actually involves surface MHC molecule binding (as opposed to non-specific binding to some other cell membrane constituent). Furthermore, live cells produce proteases that can modify cell surface MHC molecules and/or the test peptide during assay incubation., Similarly, protease inhibitors, added to the cellular assay to prevent such modifications, can themselves distort binding results. Finally, these assays do not lend themselves to high throughput, and they are less amenable to automation.

Despite the need in the art, improved methods to detect deantigenized T cell epitopes are lacking. It is critically important to the development of therapeutic proteins to be able to quickly and efficiently minimize (reduce or eliminate) an immune response in an animal to the protein therapeutic by replacing T cell epitopes of the protein therapeutic with deantigenized sequences. Therefore there is a need in the art to discover and to develop a method for detecting deantigenized T cell epitopes.

### SUMMARY OF THE INVENTION

The present invention provides a novel method for detecting a deantigenized T cell epitope. Described herein for the first time is a method for detecting a deantigenized T cell epitope, which method employs a cell solubilized or genetically produced MHC molecule (“soluble MHC” or “sMHC”) for peptide screening and binding assays. Although use of some sMHC assays to screen for peptides that induce or increase cytotoxic T lymphocyte (CTL) responses compared to a parental peptide have been described (i.e., Vitiello et al., 2001, describe hepatitis B virus surface and nucleocapsid peptide antigens with increased MHC class I-restricted CTL-stimulating properties), use of a sMHC assay to detect modified T cell epitopes having reduced or no immunogenicity (via a binding affinity to a MHC molecule less than the binding affinity of a given parental or template T cell epitope to that MHC molecule) is first contemplated and reduced to practice by the Applicant as described herein. Although reduced binding to one MHC allele is sufficient to practice the invention, reducing (or eliminating) MHC binding to multiple (i.e., two or more) MHC proteins is desirable.

Use of cell soluble MHC for binding studies to detect deantigenized T cell epitopes as taught herein provides numerous advantages over the art. Peptide binding can be characterized in real-time, employing actual biokinetic studies rather than virtual computer-generated models. Reproducibility across laboratories is much better than with cell surface assays. Substantially pure sMHC molecule solutions ensure the specificity of interaction between peptide and MHC. The opportunity for proteolytic degradation is also greatly reduced, thus reducing or eliminating the need for protease inhibitor components of the assay. Finally, sMHC assay throughput is higher than with the cell surface binding assay and is also more amenable to automation.

Soluble MHC binding assays (employing sMHC Class I molecules as well as sMHC Class II molecules), can be used to detect T cell epitopes, distinguish deantigenized T cell epitopes from T cell epitopes, and decrease immunogenicity of soluble biologic therapeutics in vivo. In addition, soluble MHC binding assays using MHC Class I and MHC Class II molecules can be used to diminish cytotoxic effector cell responses to a target antigen(s) in vivo, e.g. reducing immunogenicity of viral constructs used for gene therapy, suppressing rejection of transplanted tissues, etc.

The present invention encompasses a novel approach to develop new (modified) proteins that induce a reduced, or no, immune response (compared to an unmodified protein) in an animal (including human), wherein one or more T cell epitopes present in the unmodified protein are modified to reduce or eliminate binding to MHC molecules and thereby “short circuit” the cellular process of immunogenesis.

It is therefore an object of the present invention to provide a method for detecting a deantigenized T cell epitope by;

(a) providing an amino acid sequence of a T cell epitope having a binding affinity to a soluble MHC molecule;

(b) providing one or more altered T cell epitopes, wherein the amino acid sequence of the altered T cell epitope is different from the amino acid sequence of the T cell epitope;

(c) contacting the altered T cell epitope with the soluble MHC molecule for sufficient time to permit MHC-epitope binding complexes to form; and

(d) detecting one or more altered T cell epitopes, wherein the detected altered T cell epitope identifies a deantigenized T cell epitope having a binding affinity to the soluble MHC molecule less than the binding affinity of the T cell epitope to the soluble MHC molecule.

In one embodiment of the present invention, the method for detecting a deantigenized T cell epitope further comprises the steps of:

(e) providing one or more altered T cell epitopes, wherein the amino acid sequence of the one or more altered T cell epitopes is different from the amino acid sequence of a deantigenized T cell epitope obtained in step (d); and

(f) repeating steps (c) and (d) above.

The detected deantigenized T cell epitope need only exhibit a MHC binding affinity (measured by any of a number of methods known in the art) less than that of the parental or template T cell epitope for the present invention to be operable. Preferably, the detected deantigenized T cell epitope possesses a dissociation constant with said soluble MHC molecule greater than or equal to about 5×10⁻⁷ M. More preferably, the detected deantigenized T cell epitope possesses a dissociation constant with said soluble MHC molecule greater than or equal to about 5×10⁻⁵ M. Most preferably, the detected deantigenized T cell epitope possesses a dissociation constant with said soluble MHC molecule greater than or equal to about 5×10⁻³ M.

Similarly, detected deantigenized T cell epitope need only exhibit reduced binding (compared to the parental T cell epitope) to one MHC protein. Preferred deantigenized T cell epitopes will exhibit reduced or no binding characteristics (as described above) to at least two MHC proteins. More preferably, deantigenized T cell epitopes will exhibit reduced or no binding characteristics to multiple MHC proteins.

One aspect of the present invention is directed to a deantigenized T cell epitope detected by the methods described herein.

Once having detected a deantigenized T cell epitope, the amino acid sequence of the deantigenized T cell epitope can be substituted into the therapeutic protein in place of the parental T cell epitope, thereby rendering the modified therapeutic protein less immunogenic.

It is a further object of the invention therefore to provide a method for generating a modified polypeptide, exhibiting reduced (including no) immunogenicity compared to that of an immunogenic polypeptide (wherein the amino acid sequence of the immunogenic polypeptide has at least one T cell epitope amino acid sequence), the method comprising:

(a) detecting a deantigenized T cell epitope according to the method described above; and

(b) generating a polypeptide having an amino acid sequence modified from the immunogenic polypeptide, such that the deantigenized T cell epitope amino acid sequence detected from step (a) is substituted for the T cell epitope amino acid sequence of the immunogenic polypeptide.

Although not necessary for the operation of the present invention, preferably the modified polypeptide produced by this method exhibits a biological function similar to that exhibited by the parent immunogenic polypeptide.

A related aspect of the present invention is directed to a modified polypeptide generated by the methods described herein. Preferably the modified polypeptide exhibits reduced immunogenicity compared to that of an immunogenic (unmodified) polypeptide. More preferably, the modified polypeptide exhibits a biological function similar to that exhibited by the immunogenic polypeptide.

Preferred embodiments of the modified protein of the present invention include but are not limited to proteins that: replace endogenous proteins, exhibit enzymatic activity; act as adjuvants (including proteins capable of converting inactive drugs to active drugs in an animal); function as carriers for other molecules (e.g., proteins capable of extending the biological half-life of a molecule, including endogenous molecules, in an animal); and are capable of binding to molecules within or administered to an animal to alter the bioactivity, biodistribution and/or bioavailability of the bound molecules. More preferred modified proteins include modified immunoglobulins. Most preferred are modified monoclonal antibodies (modified Mabs). Ideally, such modified proteins, possessing deantigenized T cell epitopes, are therapeutically effective against a disease or disorder of an animal.

It is another object of the invention to provide polynucleotides that encode deantigenized T cell epitopes or modified polypeptides (containing deantigenized T cell epitopes) of the present invention. In a related aspect, the present invention includes expression vectors containing such polynucleotides, as well as host cells transformed with such expression vectors.

It is a further object of the invention to provide a pharmaceutical composition comprising a modified polypeptide generated as described herein, and pharmaceutically acceptable carrier.

Another aspect of the invention is directed to the use of the modified polypeptides of the invention to prevent, to treat, or to diagnose a disease of disorder in a vertebrate; preferably a mammal, and most preferably a human.

### DETAILED DESCRIPTION OF THE INVENTION

The present invention is directed to the detection of novel, modified T cell epitopes (deantigenized epitopes), which exhibit reduced (including absent) binding affinity to a MHC molecule compared to the binding affinity of the unmodified T cell epitope. Modified T cell epitopes exhibiting such reduced binding affinity are referred to herein as deantigenized T cell epitopes, or simply deantigenized epitopes. A T cell epitope may be identified from an antigenic polypeptide (capable of inducing an immune response in an animal) by its binding characteristics to MHC molecules. The amino acid sequence of this T cell epitope may then be used as a template to generate modified T cell epitopes, which differ from the T cell epitope by one or more amino acid residues. Modified T cell epitopes are then screened for the ability to bind MHC molecules. Any modified T cell epitope exhibiting a MHC binding affinity lower than that exhibited by the (template or parental) T cell epitope defines a deantigenized T cell epitope. Once a deantigenized epitope is detected, the amino acid sequence of the deantigenized epitope can be substituted for the T cell epitope into the antigenic polypeptide, thereby reducing or eliminating the immunogenicity of the antigenic polypeptide.

Because the vertebrate cellular immune response to specific antigens involves two steps; presentation of a peptide from the antigen by MHC molecules, and recognition of this complex by antigen-specific receptors of immune cells, alteration of either of these steps will alter the immune response. The MHC of higher vertebrates (e.g., mammals; also referred to as Human Leucocyte Antigen, HLA, in humans) is central to the presentation process, and thus plays an essential role in regulating the immune system.

MHC proteins, which are expressed in a vertebrate cell in multiple allelic forms, form complexes with antigenic peptides, and are displayed on the surface of the cell where they are recognized by T cells. Upon recognition of the MHC-peptide complex, the T cell receives an activation signal through the antigen-specific receptor that induces a T cell response (e.g., T cell proliferation and cytokine production), thus starting the immune response. Although reduced binding of a deantigenized T cell epitope to one MHC allelic form is sufficient to practice the invention, reducing (or eliminating) MHC binding to multiple (i.e., two or more) MHC proteins is preferred.

There are two classes of MHC proteins, known as MHC class I and MHC class II. They are similar in that they both form a binding groove for complexing with antigenic peptides, and both form antigen peptide complexes for presentation of an antigen in a conformation recognizable by specific T cells for induction of an immune response. Peptides that complex with MHC molecules (i.e, “T cell epitopes” as used herein) are typically about eight to about twenty-four amino acids in length.

MHC class I proteins are expressed in all nucleated cells of higher vertebrates. MHC class I molecules generally bind peptides derived from endogenous antigens (e.g., normal, “self” cellular proteins, or viral or bacterial proteins produced within an infected cell), which have been processed within the cytoplasm of the cell (the cytosolic pathway). MHC class I antigen complexes, properly displayed on the surface of the cell are typically recognized by cytotoxic T cells (T_{c}, specifically CD8⁺ cells). Presentation of an endogenous or “self” peptide by the MHC class I antigen complex does not typically elicit a T cell response since, under normal circumstances, cytotoxic T cells that would otherwise recognize the surface complex and attack the presenting cell have been eliminated (deleted) from the immune system repertoire (one method of inducing “tolerance”). Presentation of “foreign” (“non-self” such as viral) peptides by the MHC class I antigen complex elicits cytotoxic T cell attack and cytolytic destruction of the infected or diseased cell. Thus, MHC class I antigen complexes either mark the cell as a normal endogenous cell, which elicits no immune response, or mark the cell as an infected cell (e.g., as in the case of a virus-infected cell, exhibiting intracellularly processed viral peptide in the surface MHC class I antigen complex) or a transformed cell (e.g., such as a malignant cell), which elicits attack on the diseased cell by cytotoxic T cells.

MHC class II proteins are expressed in a subset of nucleated vertebrate cells, conventionally referred to as Antigen Presenting Cells, or “APCs”. MHC class II molecules generally bind peptides derived from exogenous antigens, which are internalized by phagocytosis or endocytosis and processed within the endosomal/lysosomal pathway. MHC class II antigen complexes, properly displayed on the surface of an APC are recognized by helper T cells (Tₕ, specifically CD4⁺ cells). Helper T cell recognition results in release of lymphokines and T-dependent activation of B cells, which, in turn, lead to activation of macrophages and release of antibodies from B cells (respectively), leading to the killing or elimination of invading microorganisms. The immune recognition events mediated by MHC class II antigen complexes are a primary defense to invading microorganisms (e.g., bacteria, parasites) or foreign substances (e.g., haptens, transplant tissues) introduced to the cells of the immune system via the circulatory or lymph systems. Many of the CD8+Tc responses are also dependent on initial help from CD4+Th cells.

The present invention is useful to reduce or to eliminate T cell based immunogenicity of all types of peptide-containing molecules, without limitation that are in some way or for some reason immunogenic when administered to an animal. These include all forms of naturally occurring, recombinant, chimeric, and fusion proteins, lipoproteins, glycoproteins, or modified proteins detected or derived from all Domains of life: Bacteria, Archaea, and Eukarya. Potentially therapeutic proteins are of particular interest, especially animal-derived proteins, and most especially human or humanized proteins (e.g., antibodies).

Following the convention of practitioners skilled in the art, the following illustrative explanations are provided to facilitate understanding of certain terms and phrases frequently used and of particular significance herein.

Peptide, oligopeptide, polypeptide, and protein refer to any polymer of two or more amino acid residues, typically L-amino acids, connected one to the other typically by peptide bonds between the alpha-amino and carbonyl groups of adjacent amino acids.

T cell epitope, as used herein, refers to any peptide sequence with the ability to bind MHC molecules or functional fragments thereof (e.g., sMHC molecules). As such, “T cell epitope” is operationally equivalent to an “MHC binding peptide”, as known in the art. Use of the term “T cell epitope” is preferred only to emphasize that by detecting modified peptide sequences, which exhibit reduced binding affinity to MHC, the modified peptide sequence also, inherently, is less immunogenic (“deantigenized”) as compared to the unmodified peptide sequence. T cell epitope sequences of the present invention preferably are used as “templates” or “parental” sequences for the generation of modified T cell epitopes, some of which will be deantigenized T cell epitopes by virtue of their reduced binding affinity to MHC. Both parental and modified T cell epitopes may be characterized and quantified by any computational or physical method useful to establish MHC binding. Although implicit in the term “T cell epitope” is the ability of the peptide sequence to be recognized by a T Cell Receptor (TCR) and thereby (in theory) induce T cell activation, TCR binding is not a necessary feature of the T cell epitope of the present invention.

A conserved residue (e.g., of a given T cell epitope) is an amino acid that occurs in a significantly higher frequency than would be expected by random distribution at a particular position in a peptide motif. Typically, a conserved residue of a T cell epitope identifies a contact point with a MHC molecule (e.g., in close contact with the MHC peptide binding groove, with its side chain buried in a specific pocket of the groove itself). A T cell epitope preferably possesses one to about three conserved residues. These residues are the preferred sites of T cell epitope modification as part of the deantigenizing process.

Deantigenize refers to a process of reducing or eliminating the immunogenicity of a peptide, oligopeptide, polypeptide, or protein. As discussed, the immune response requires i) T cell epitope presentation (by a MHC molecule), and ii) TCR recognition of the T cell epitope resulting in T cell activation. Reduction in the efficiency of either process will result in a reduction in the immunogenicity of the T cell epitope (and, therefore, polypeptides containing such T cell epitopes). It is an object of the present invention to reduce immunogenicity of a peptide by reducing T cell epitope binding affinity to MHC. Deantigenizing is accomplished in the present invention by detecting a deantigenized T cell epitope, wherein the deantigenized T cell epitope has an amino acid sequence or amino acid modification (e.g. glycosylation) different from a (parental) T cell epitope and exhibits a reduced binding affinity to an MHC molecule (compared to the MHC binding affinity of the unmodified T cell epitope). Deantigenized peptides may refer to a deantigenized T cell epitope, itself (as described above), or to a larger modified polypeptide, wherein one or more T cell epitopes of the larger polypeptide have been replaced with one or more deantigenized T cell epitopes. Particularly preferred modified polypeptides of the present invention include modified antibodies, wherein one or more T cell epitopes of the antibody have been replaced by deantigenized T cell epitopes. Preferred modified polypeptides of the present invention will exhibit reduce immunogenicity in an individual compared to its corresponding unmodified polypeptide.

Detection, as used herein, broadly refers to any technique or mechanism known in the art whereby a particular chemical species is discernible (quantitatively or qualitatively) from other chemical species existing in a solution. Detection includes isolation techniques (i.e., the substantial or reasonable separation of molecules from other molecules) from solution. Detection methods may include, but are not limited to, any molecular or cellular techniques, used singularly or in combination, including, but not limited to: hybridization and/or binding techniques, including blotting techniques and immunoassays; labeling techniques (chemiluminescent, colorimetric, fluorescent, radioisotopic); spectroscopic techniques; separations technology, including precipitations, electrophoresis, chromatography, centrifugation, ultrafiltration, cell sorting; plasmon resonance; and enzymatic manipulations (e.g., digestion).

Transfection, as use herein, is defined broadly and intended to encompass any technique useful for the introduction of exogenous DNA into a cell (prokaryotic or eukaryotic).

Host cell (or recombinant host cell), as used herein, is refers to any cell (prokaryotic or eukaryotic) into which a recombinant genetic vector may be introduced. Particularly for generating modified polypeptides of the invention, the host cell is preferably a eukaryotic cell (more preferably a yeast or mammalian cell) because eukaryotic cells are more likely than prokaryotic cells to assemble and secrete a properly folded, glycosylated, and biologically active protein (e.g., antibodies). Particularly preferred mammalian host cells for expressing, for example, modified antibodies of the present invention include Chinese Hamster Ovary (CHO) cells (Urlaub and Chasin, 1980; Kaufman and Sharp, 1982). It is also understood that host cell refers not only to a particular subject cell engineered to include a genetic vector, but also to the progeny of such a cell. Because genetic modifications may occur in succeeding cell generations (e.g., natural mutational or recombinatorial phenomena), such progeny may not, in fact, be genetically identical to the parent cell, but are still included within the scope of the term “host cell” as used herein.

Preventing, or treating a disease or disorder generally refers to any process that functions to slow, to halt (including stopping initial onset), or to reverse any adverse clinical or pathological symptom exhibited by an individual. Diagnosing a disease or disorder refers to any procedure useful to detect (in vivo or ex vivo) the presence of a disease or disorder in an individual.

Therapeutically effective amount is an amount effective to achieve a desired physiological result in a subject. For example, a therapeutically effective amount of a therapeutic antibody is an amount sufficient to alter (enhance or hinder) the biological effects of the target antigen for such an antibody for a period of time sufficient to ameliorate one or more of the pathological processes associated with the biological activity of that target antigen. The effective amount will vary depending on the specific therapeutic agent selected and its mode of delivery. A therapeutically effective effective amount is also dependent on a variety of factors and conditions related to the subject to be treated (for example, age, weight, sex, and health of the patient, as well as dose response curves and toxicity data) and the severity of the disorder, and includes within its definition “prophylactically effective amounts” (preventative or pre-clinical treatment may require lesser dosage than later stage or late stage conditions) and “remission maintenance” amounts.). Determination of a therapeutically effective amount for a given agent is well within the ability of those skilled in the art.

Pharmaceutically acceptable carrier, as used herein and generally known in the art, refers to any and all solvents, dispersion media, coatings, antibacterial and antifungal agents, isotonic and absorption delaying agents, carrier proteins, and the like that are physiologically compatible in an individual. Examples of pharmaceutically acceptable carriers include one or more of water, saline, phosphate buffered saline, bovine serum albumin (BSA), dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion. Physiologically acceptable salt forms and standard pharmaceutical formulation techniques are well known to persons skilled in the art (see, for example, Remington's Pharmaceutical Sciences, Merck Publishing Co.).

Administration to an individual is not limited to any particular delivery system and may include, without limitation, parenteral (including subcutaneous, intravenous, intraarticular, intramuscular, or intraperitoneal injection), rectal, topical, nasal, inhalation, transdermal or oral (for example, in capsules, suspensions or tablets) delivery. Administration to an individual may occur in a single dose or in repeat administrations, and in any of a variety of physiologically acceptable salt forms, and/or with an acceptable pharmaceutical carrier as part of a pharmaceutical composition. Administration of a therapeutic agent to an individual may also be by means of gene therapy, wherein a nucleic acid sequence encoding the agent is administered to the individual in vivo, or to cells in vitro (which are then introduced into an individual); the agent is produced by expression of the product encoded by the nucleic acid sequence. Methods for gene therapy are also well known to those of skill in the art.

As used herein, an “individual” refers to any animal, preferably mammal, most preferably human, subject that may be afflicted with or potentially susceptible to a disease or disorder.

### DESCRIPTION OF THE PREFERRED EMBODIMENTS

Based upon the discoveries reported here for the first time, the present invention is generally directed to a method for detecting a deantigenized T cell epitope by;

(a) providing an amino acid sequence of a T cell epitope having a binding affinity to a soluble MHC molecule;

(b) providing one or more altered T cell epitopes, wherein the amino acid sequence of the altered T cell epitope is different from the amino acid sequence of the T cell epitope;

(c) contacting the altered T cell epitope with the soluble MHC molecule for sufficient time to permit MHC-epitope binding complexes to form; and

(d) detecting one or more altered T cell epitopes, wherein the detected altered T cell epitope identifies a deantigenized T cell epitope having a binding affinity to the soluble MHC molecule less than the binding affinity of the T cell epitope to the soluble MHC molecule.

Furthermore, once detected, the amino acid sequence of the deantigenized T cell epitope can be substituted into an immunogenic polypeptide (e.g., a therapeutic protein possessing the T cell epitope) in place of the existing T cell epitope, thereby producing a modified polypeptide, which is less immunogenic than the immunogenic polypeptide.

### I. Providing a T Cell Epitope

The present invention is not limited by any method or technique of providing (e.g., identifying) a T cell epitope; any method of identifying a T cell epitope is useful in the present invention. Present methods known in the art include but are not limited to the use of: animal models, including transgenic animals such as transgenic mice expressing human MHC molecules (Taneja and David, 1998); T cell assays, including peripheral blood mononuclear cell (PBMC) assays (Stickler et al., 2000); MHC binding assays, including soluble MHC class I and soluble MHC class II assays, such as HLA-DR and HLA-DQ binding assays (see below); and predictive, comparative, or computational modeling of MHC binding (e.g., Delhaise et al., 1984; Devereux et al. 1984).

As used herein, a T cell epitope essentially is capable of binding a MHC molecule. Sette et al. (1989) showed that MHC allele specific motifs could be used to predict MHC binding capacity. Schaeffer et al. (1989) showed that MHC binding was related to immunogenicity. It has been demonstrated that MHC class I binding motifs can be applied to the identification of potential immunogenic peptides in animal models (e.g., De Bruijn et al., 1991; Pamer et al., 1991).

A preferred method of providing a T cell epitope, therefore, involves computational methods of identifying MIHC class I or class II binding peptides, such as computational “threading” algorithms (e.g., see Altuvia et al., 1995). For example, computer analysis may be conducted using MPT (ver 1.0) software (Biovation, Aberdeen, UK), which performs peptide threading according to the methods disclosed by Fothergill et al., 1998, wherein, an index of potential peptide binding to 18 different MHC class II DR alleles (covering greater than 96% of the HLA-DR allotypes extant in the human population) may be calculated. Alternatively, or in concert, a comparison of suspected epitope sequences may be made against preexisting databases of MHC-binding motifs (e.g., “MHCPEP: A database of MHC binding peptides, v.1.3”, Brusic, 1998, http://wehih.wehi.edu.au/mhcpep/).

One preferred process for identifying and utilizing deantigenized T cell epitopes of, for example, an antibody immunogenic to an individual is as follows:

Identify sequences in the antibody that are different from the germline sequence.

These sequences are usually in, but not restricted to, the complementarity determining region (CDR) or hypervariable regions of the heavy and light chain variable regions, and the allotypes in the constant region of the heavy chain.

These regions are the most likely to be immunogenic in vivo.

Generate multiple overlapping synthetic peptides (preferably about 13 to about 25 amino acid residues in length), preferably offset or staggered by 1-2 amino acids, and which extend into the identified sequence above by at least one amino acid.

Test the affinity of interaction of the above overlapping peptides to sMHC molecules (MHC Class I or II) by methods known in the art (including but not limited to direct binding assays in solution phase by energy transfer, using analytes labeled by radioactive or other means such as biotin, competition assays using labeled analytes, plasmon resonance (e.g., BIACORE International AB, Upsala, Sweden), or other methods.

Identify peptides exhibiting moderate to high binding affinity to the sMHC molecule (preferably having, for example, Kd<1000 nM), wherein said peptides identify T cell epitopes of the immunogenic antibody.

### II. Providing an Altered T Cell Epitope

Once a T cell epitope has been identified, an altered T cell epitope is provided, wherein the amino acid sequence of the altered T cell epitope is different from the amino acid sequence of the (parental) T cell epitope. According to the invention, only one altered T cell epitope need be provided. Preferably, a plurality of altered T cell epitopes (e.g., “a library”) is provided.

Methods of T cell epitope alteration include any form of chemical alteration of a given epitope polypeptide, including but not limited to amino acid sequence alteration, glycosylation, and covalently linked lipid phophorylation modifications. Particularly preferred methods for T cell epitope alteration include random and nonrandom amino acid sequence changes to a given T cell epitope. Nonrandom alteration includes but is not limited to: altering one or more conserved amino acid residues of the T cell epitope; altering one or more residues adjacent (in terms of primary structure) to conserved residues; or altering one or more residues not adjacent to conserved residues, but which may be in conformational proximity (in terms of secondary structure) to a conserved residue. A library of altered T cell epitopes with single amino acid substitutions may be generated to determine the effect of electrostatic charge, hydrophobicity, etc. on binding. For example, positively charged (e.g., Lys or Arg) or negatively charged (e.g., Glu) amino acid substitutions may be made along the length of the peptide, revealing different MHC binding affinities. Multiple substitutions, using small, relatively neutral moieties (e.g., Ala, Gly, and Pro) may be employed. Alterations may involve homo-oligomers or hetero-oligomers. The number and types of residues that are substituted or added may depend on steric considerations as well as functional attributes of the epitope (e.g., hydrophobicity versus hydrophilicity).

T cell epitope alteration preferably refers to residue substitution in the amino acid sequence of a T cell epitope, but may also include amino acid deletion and/or addition. The T cell epitope can also be altered by extending or decreasing its amino acid sequence, or modifying amino acid residues, e.g. glycosylation.

A multitude of methods, techniques, and kits for generating libraries of variant polypeptides is well known in the art and commercially available. Such methods include but are not limited to genetic engineering, recombinant nucleic acid technology, protein chemistry, or any other means of molecular synthesis or alteration. One preferred method includes site directed mutagenesis of synthetically produced oligonucleotides encoding the parental T cell epitope (e.g., using the QUICK-CHANGE protocol and reagents of Stratagene, Cambridge, UK). Similar to the initial step of providing a T cell epitope, the present invention is not limited the method or technique of providing altered T cell epitopes; any method of altering a T cell epitope is useful in the present invention.

One preferred process for identifying and utilizing deantigenized T cell epitopes of, for example, an antibody immunogenic to an individual continues as follows:

Modify the T cell epitope identified above in an iterative manner by amino acid substitution, such that the modified T cell epitope exhibits a lower affinity to the MHC molecules compared to that of the unmodified T cell epitope (as measured by one or more of the sMHC binding assays), wherein said modified T cell epitope identifies a deantigenized T cell epitope.

### III. Soluble MHC (sMHC) Assays

Central to the practice of the present invention is the use of a soluble MHC (class I or class II) assay to detect altered T cell epitopes having a binding affinity to the soluble MHC molecule less than the binding affinity of the T cell epitope to the soluble MHC molecule (the “contacting” and “detecting” steps of the present invention). The use of in vitro sMHC assays as taught in the present invention enables the skilled practitioner readily to identify (and to quantitate) deantigenized epitopes from undesirable altered T cell epitopes (those that exhibit the same or an increased MHC binding affinity), and it is critical to the present invention.

The preparation of sMHC molecules is well known in the art; e.g., by immunoprecipitation, affinity chromatography, ion exchange chromatography, lectin chromatography, size exclusion, high performance ligand chromatography, or a combination of thereof, from an appropriate cell line. Particular cell lines from which MHC molecules may be isolated are nonlimiting and known in the art (including but not limited to human EBV-transformed B cell lines, or cell lines transfected with specific HLA genes useful for isolation of either MHC class I or class II molecules). Several lines useful for the preparation of sMHC molecules are known to the skilled practitioner and publicly available (e.g., American Type Culture Collection, Rockville, Md.; National Institute of General Medical Sciences Human Genetic Mutant Cell Repository, Camden, N.J.; Brigham & Women's Hospital ASHI Repository, Boston, Mass.).

A variety of techniques and reagents generally used to perform the sMHC assay component of the present invention are also well known in the art, and include assays to sMHC panels (e.g., see methods disclosed by Falk et al., 1991; Kubo et al., 2000; and Vitiello et al., 2001, incorporated herein by reference). Specific techniques and protocols to detect and to quantitate MHC binding of altered T cell epitopes are chosen based upon practitioner needs or preferences. Altered T cell epitopes-sMHC molecule elution/separation techniques may include but are not limited to acid elution, reversed-phase high performance liquid chromatography (HPLC), ion exchange chromatography, size chromatography, filtration, electrophoresis, immunoprecipitation, and any of a variety of denaturing techniques (heat, pH, detergents, salts, chaotropic agents, or a combination thereof).

Once one or more deantigenized T cell epitopes have been identified, their amino acid sequence may be determined (if not known a priori) or confirmed using techniques well known in the art, e.g., Edman degradation, mass spectrometry (e.g., Hunkapiller et al., 1983; Hunt et al., 1992). Comparison of multiple deantigenized T cell epitopes may reveal generalized or conserved deantigenizing motifs useful for a given epitope and/or given MHC molecule.

The method described above enables the operable screening protocol essential for detecting a deantigenized T cell epitope. Further embodiments of this method are also contemplated and within the scope of the present invention, however. For example, additional rounds of epitope modification and sMHC screening may be desirable in an effort to detect additional or improved deantigenized T cell epitopes. In addition, deantigenized T cell epitopes may be modified further in an effort to add desirable chemical or biological features to the peptide or to remove undesirable features. Such chemical modifications are well known to those skilled in the art, e.g., glycosylation, side chain oxidation, phosphorylation, etc (additional modifications are discussed elsewhere in this disclosure).

### IV. Generating a Reduced Immunogenic Polypeptide

A further aspect of the present invention is to provide a polypeptide, modified from an immunogenic polypeptide, such that one or more T cell epitopes of the immunogenic polypeptide is/are replaced with deantigenized T cell epitope(s). The resulting modified polypeptide is thereby rendered less immunogenic than the original immunogenic polypeptide. According to the present invention, once one (or more) deantigenized T cell epitope has been identified, immunogenic polypeptides, which typically possess the T cell epitope may be modified by altering the amino acid sequence of the T cell epitope to that of the deantigenized T cell epitope, thereby rendering the modified polypeptide less immunogenic than its unmodified, immunogenic parent.

As used herein, epitope “substitution” or “replacement” (e.g., a deantigenized T cell epitope amino acid sequence substituted for a T cell epitope amino acid sequence) within a polypeptide may be, but is not limited to, actual physical translocation of the peptide sequences; i.e., deletion and insertion of the appropriate parental and deantigenized T cell epitopes. Epitope substitution may be achieved by any of a number of techniques known in the art to effect amino acid sequence changes. These include but are not limited to de novo synthesis of the modified polypeptide and/or nucleic acid encoding the modified polypeptide as well as any manipulation of the nucleic acids that encode the immunogenic polypeptide (or T cell epitopes found therein), including any recombinant nucleic acid technique further including nucleic acid mutagenesis (e.g., point mutations).

Amino acid substitutions typically are single residue substitutions. Substitutions, deletions, insertions, or any combination thereof may be combined to arrive at a final peptide. Substitutional variants are those in which at least one residue of a T cell epitope has been removed and replaced with a different residue. Such substitutions are well known to persons skilled in the art.

Modified polypeptide, having one or more deantigenized T cell epitopes, may be further modified (as determined by the practitioner) to provide additional desirable attributes, e.g., improved pharmacological characteristics, yet retaining at least some of the reduced immunogenic attributes of the modified polypeptide. For instance, the peptides may be subject to various changes, such as substitutions, either conservative or nonconservative, where such changes might provide for certain advantages in their use, such as improved bioavailability or increased bioactivity. Conservative substitutions refer to replacing an amino acid residue with another, which is biologically and/or chemically similar (e.g., hydrophobic residues, polar residues, negatively charged residues, etc.). Such conservative substitutions are well known and understood in the art.

Preferred modified polypeptides are potentially therapeutic to treat a disease or disorder of an individual. Modified polypeptides of the present invention may be non-autologous or autologous. They include without limitation: proteins that replace, substitute, or augment endogenous proteins (e.g. hormones, cytokines, growth factors); proteins having an enzymatic activity; proteins capable of converting an inactive compound (i.e., drug) to an active compound, or visa versa, within an individual; proteins functional as carriers for other molecules within an individual; and proteins that bind to other molecules within, or introduced within, an individual in order to alter the bioactivity, bioavailability, or biodistribution of the other molecules.

Preferably, an immunogenic polypeptide is rendered less immunogenic by the replacement of one or more T cell epitopes with deantigenized T cell epitopes, without loss of the biological activity of the polypeptide. It is desirable, therefore, that the modified polypeptide is tested not only for its reduced immunogenicity, but for its biological activity as well.

One preferred process for identifying and utilizing deantigenized T cell epitopes of, for example, an antibody immunogenic to an individual continues as follows: Replace the identified T cell epitope sequence in the native antibody molecule with the identified deantigenized T cell epitope.

### V. Validating a Reduced Immunogenic Polypeptide

In addition to the method steps described above (including optional, additional rounds of deantigenizing screening), further testing steps may be part of the present invention. In one embodiment, the modified polypeptide may be tested to confirm reduced ability of the modified polypeptide to activate the various component systems of an immune response. Numerous MHC and T cell activation assays are available and well known in the art, some of which have been discussed earlier in this disclosure, including but not limited to PBMC assays, which measure the ability to activate T cells and induce proliferation of T lymphocytes (e.g., as present in the circulation of humans). These assays include, but are not limited to, proliferation assays, cytokine release, gene expression by PCR techniques, and calcium flux. Such assays exemplify an ex vivo assay, which provides the practitioner insight in the potential effects of the modified polypeptide on in vivo immunogenicity. A variety of animal model protocols are also known in the art. For example, immunogenicity can also be tested in vivo in animals genetically engineered to express human MHC molecules, or in primates, using standard methods such as T cell activation assays in cells removed from animals administered these proteins/peptides, and/or by studying antibody responses in these animals.

Furthermore, after the replacement of one or more T cell epitopes with deantigenized T cell epitopes of the present invention, the modified polypeptide may be tested for some desired activity. Preferably, the modified polypeptide retains sufficient activity to be biologically (e.g., therapeutically) useful. More preferably, the modified polypeptide retains at least about half (50%) activity as compared to the immunogenic polypeptide. More preferably, the modified polypeptide retains at least about 75%, 80%, 85%, or 90% activity as compared to the immunogenic polypeptide. Most preferably, any loss of activity of the modified polypeptide as compared to the immunogenic polypeptide is statistically insignificant. In some circumstances, the activity of the modified polypeptide may be advantageously altered from that of the immunogenic polypeptide.

It is understood by those skilled in the art that testing the activity of modified proteins (e.g., validating the biological activity of the modified polypeptide) is highly specific to the nature of immunogenic polypeptide, which has been so modified. For example, an antibody, which has been modified by substitution with one or more deantigenized T cell epitopes of the present invention, may be validated by comparing the binding affinity of the modified antibody and immunogenic antibody to a given antigen, or by comparing the respective activities in a bioassay specific for the target antigen. The nature and extend of validating experimentation of a modified polypeptide of the present invention is case specific and at the preference, selection, and discretion of the practitioner.

Therefore, one preferred process for identifying and utilizing deantigenized T cell epitopes of, for example, an antibody immunogenic to an individual continues as follows:

Assay the biological activity of the modified antibody generated above. If the activity is substantially lower than the native antibody, revert to the original sequence, substituting other residues and other positions until a modified antibody is obtained having lower binding affinity to sMHC yet retaining sufficient biological activity. The resulting modified antibody represents a functional antibody having reduced immunogenicity.

Optionally;

The immunogenicity of the native antibody, T cell epitopes identified from the native sequence, deantigenized T cell epitopes, and the full length modified antibody, may be tested in vitro using standard human T cell activation assays using cells from peripheral blood or other sources, and/or using in vivo animal models.

### VI. Producing a Reduced Immunogenic Polypeptide

The various polypeptide embodiments of the invention can be prepared in a wide variety of ways. Polypeptides can be synthesized in solution or on a solid support in accordance with conventional techniques. Various automatic synthesizers are commercially available and can be used in accordance with protocols well known in the art (e.g., Stewart and Young, 1984).

Preferably, recombinant nucleic acid technology is utilized. The present invention also relates to methods for producing deantigenized T cell epitopes and modified polypeptides of the invention by preparing nucleic acids that encode at least a functional domain thereof (i.e., the part of the structural information of the epitope or polypeptide that provides for its biological activity); cloning the nucleic acids in a vector; transforming or transfecting a host cell with the vector, and culturing the host cell under conditions suitable for expressing the nucleic acid. These procedures are all well known in the art, as described generally in a variety of molecular biology manuals, some of which are cited herein; all of which are incorporated herein by reference.

In one example, the coding sequence for a deantigenized T cell epitope of the present invention is especially suitable for synthesis following known chemical techniques. The coding sequence may be provided with appropriate linkers and ligated into any of a multitude of publicly available expression vectors. The deantigenized T cell epitope may be operably inserted directly into a vector, or operably inserted into a vector containing the immunogenic polypeptide, thereby replacing a T cell epitope of that immunogenic polypeptide with its deantigenized T cell epitope counterpart. The vectors, in turn, may be used to transform suitable hosts to produce the polypeptide. Once again, a variety of expression vectors and host systems (including bacterial, fungal, protist, plant, and animal host systems) are well known, readily available, and within the skill of practitioners in the art.

Reduced Immunogenic Antibodies

Particularly preferred modified polypeptides of the present invention include modified antibodies, wherein one or more identified T cell epitopes have been replaced with (corresponding) deantigenized T cell epitopes according to the present invention. Such antibodies are particularly useful as diagnostic or therapeutic agents.

An antibody (including functional antibody fragments), of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies well known in the art may be use to generate and to isolate deantigenized antibody heavy and light chain genes or genes encoding deantigenized therapeutic proteins of the present invention, to incorporate such genes into expression vectors, to transfected host cells with such expression vectors, and to isolate the modified antibody from such transfected host cells.

Modified antibodies of the present invention also include functional fragments thereof, wherein the antibody fragment retains the ability to bind a particular antigen. Preferred antibody fragments include Fab, F(ab)₂, scFv, diabody, and single domain antibody molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to a given antigen. In addition, bifunctional (e.g., bi-specific or dual specific) antibodies are within the scope of the present invention.

### VI. Reduced Immunogenic Polypeptide Uses

Because modified polypeptides of the present invention include those that have potential clinical or therapeutic benefit when administered to an individual, a further aspect of the present invention is directed to use of the modified polypeptide to diagnose, to treat, or to prevent a disease or disorder of an individual. Modified polypeptides of the present invention are especially advantageous for such applications because they exhibit reduced immunogenicity in an individual compared to the unmodified polypeptide, especially when such applications involve multiple administration of the modified polypeptide to an individual.

### VI. Reduced Immunogenic Polypeptide Compositions and Administration Thereof

Modified polypeptides of the present invention can be incorporated into pharmaceutical compositions suitable for administration to an individual. Typically, the pharmaceutical composition comprises modified polypeptide of the invention and a pharmaceutically acceptable carrier. Pharmaceutical compositions of this invention may be in a variety of forms known in the art. These include, for example, liquid, semi-solid and solid dosage forms, such as liquid solutions (e.g., injectable and infusible solutions), dispersions or suspensions, tablets, pills, powders, aerosols, liposomes and suppositories. The preferred form depends on the intended mode of administration, therapeutic application, and desired affects. For modified antibodies of the present invention, preferred compositions are in the form of injectable or infusible solutions, such as compositions similar to those used for passive immunization with other antibodies. The preferred mode of administration is parenteral (e.g., intravenous, subcutaneous, intraperitoneal, intramuscular). In a preferred embodiment, the modified antibody is administered by intravenous infusion or injection. In another preferred embodiment, the antibody is administered by intramuscular or subcutaneous injection.

In certain embodiments, a modified polypeptide of the invention may be intranasally or orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be appropriate to coat the compound with, or co-administer the compound with, a material to prevent its inactivation.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., modified antibody or antibody fragment) in the required amount in an appropriate solvent with one or more of a combination of ingredients enumerated above, as required, followed by filtered sterilization and/or gamma irradiation. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants.

Modified polypeptides of the present invention can be administered by a variety of methods known in the art, although for many therapeutic applications, the preferred route/mode of administration is intravenous injection or infusion. As will be appreciated by those skilled in the art, the route and/or mode of administration will vary depending upon the desired effects. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Prolonged absorption of injectable compositions can be brought about by including in the composition an agent that delays absorption, for example, monostearate salts and gelatin. Many methods for the preparation of such formulations are patented or generally known to those skilled in the art (e.g., see Robinson, 1978).

Supplementary active compounds can also be incorporated into the compositions. In certain embodiments, a modified polypeptide of the invention is coformulated with and/or coadministered with one or more additional therapeutic agents that are useful for treating a particular disease or disorder. Such combination therapies may advantageously utilize lower dosages of the administered therapeutic agents, thus avoiding possible toxicities or complications associated with the various monotherapies.

Dosage regimens may be adjusted to provide an optimum desired response. For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the individual to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic or prophylactic effect to be achieved, and the limitations inherent in the art of compounding such an active compound for the treatment of sensitivity in individuals.

The present invention incorporates by reference in their entirety techniques well known in the field of molecular biology. These techniques include, but are not limited to, techniques described in the following publications:

Ausubel, F. M. et al. eds., Short Protocols In Molecular Biology (4th Ed. 1999) John Wiley & Sons, NY. (ISBN 0-471-32938-X).

Fink & Guthrie eds., Guide to Yeast Genetics and Molecular Biology (1991) Academic Press, Boston. (ISBN 0-12-182095-5).

Kay et al., Phage Display of Peptides and Proteins: A Laboratory Manual (1996) Academic Press, San Diego.

Lu and Weiner eds., Cloning and Expression Vectors for Gene Function Analysis (2001) BioTechniques Press. Westborough, Mass. 298 pp. (ISBN 1-881299-21-X).

Old, R. W. & S. B. Primrose, Principles of Gene Manipulation: An Introduction To Genetic Engineering (3d Ed. 1985) Blackwell Scientific Publications, Boston. Studies in Microbiology; V.2:409 pp. (ISBN 0-632-013184).

Robinson ed., Sustained and Controlled Release Drug Delivery Systems (1978) Marcel Dekker, Inc., NY.

Sambrook, J. et al. eds., Molecular Cloning: A Laboratory Manual (2d Ed. 1989) Cold Spring Harbor Laboratory Press, NY. Vols. 1-3. (ISBN 0-87969-309-6).

Stewart and Young, Solid Phase Peptide Synthesis (2d. Ed. 1984) Pierce Chemical Co.

Winnacker, E. L. From Genes To Clones: Introduction To Gene Technology (1987) VCH Publishers, NY (translated by Horst Ibelgaufts). 634 pp. (ISBN 0-89573-614-4).

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention.

### EXAMPLE 1

Identification of Anti-IL-12 Monoclonal Antibody (MAb) T Cell Epitopes

To identify T cell epitopes of an anti-IL-12 monoclonal antibody (MAb), human PBMCs are tested for their reactivity using the parent anti-IL-12 antibody and antibody derived peptides using techniques well known in the art (Stickler et al., 2000). Briefly, peptides, 13 residues in length and staggered one residue at a time, are synthesized using a fluorenylmethoxycarbonyl-protected amino acid coupling procedure (Hiemstra et al., 1997). T-cells are tested for reactivity by co-culturing the peripheral blood derived T-cells, mitomycin C-treated autologous EBV-B-cells or other antigen presenting cells such as dendritic cells, and the antibody or the appropriate peptide for 4 days. The T cells are pulsed for the last 24 hours with tritiated thymidine, harvested, and analyzed for thymidine uptake, thus determining cell proliferation after stimulation with the MAb or a specific peptide. Cell proliferation of at least two times the background, reveals specific reactivity of the T cells, and the presence of distinct T cell epitopes within the MAb .

### EXAMPLE 2

sMHC Assay for Confirming MAb T Cell Epitopes

To demonstrate that the MAb T cell epitope sequences identified as described in Example 1 are capable of binding to an appropriate MHC class II molecule and to establish a measure of sMHC binding affinity as a basis for comparison to alter T cell epitope binding affinity, specific binding assays are employed as described in Kubo et al, 2000 (the entirety of which is incorporated herein by reference).

Briefly, HLA-DR and DQ molecules are purified, modified to allow for radioiodination, and labeled by the use of the Chloramine T method (Buus et al., 1987). Labeled HLA is incubated for two days with 10 nM of synthesized MAb T cell epitope from Example 1 at pH 7.0 and 23° C., preferably in presence of a protease inhibitor cocktail (1 mM PMSF, 1.3 mM 1.10 phenanthroline, 73 μM pepstatin A, 8 mM EDTA, and 200 μM N aₚ-tosyl-L-lysine chloromethyl ketone (TLCK)). Bound radioactivity is measured by gel filtration over TSK 2000 columns (e.g., see Sette et al., 1991) and Kd calculated by standard procedures.

Variants of this protocol are also used to test binding of large numbers of synthetic anti-IL-12 antibody T cell epitopes to a variety of other MHC class II specificities as previously cited (e.g., HLA-DQ2, HLA-DR4). Binding affinities determined from the sMHC assays for each epitope tested are used as the basis of comparison for selecting deantigenized MAb T cell epitopes (see Example 4 below).

### EXAMPLE 3

Altering a MAb T Cell Epitope

Data derived from Example 2 confirm sMHC-T cell epitope binding. To determine that specific residue positions of the MAb T cell epitope are necessary for MHC binding and to demonstrate that alteration of the epitopes by removing or replacing these residues reduces or eliminates MHC binding, MAb T cell epitope analogues are generated.

As described earlier, computer modeling techniques, capable of predicting interaction energy of amino acid sequences within the MHC binding groove, is used to model sequences that are potentially less favorable for binding at each position in the peptide-binding groove. Since interaction energy is interpreted as a measure of an amino acid involvement in MHC binding, less well binding residues lower the affinity or even disrupt the binding of the peptide to the MHC molecule. (Alternatively, peptides with random or sequential mutations in the parent T cell epitope are used to detect peptides of lower binding affinity to the sMHC molecules.)

Altered MAb T cell epitopes are generated by solid phase strategies well known in the art on a multiple peptide synthesizer by repeated cycles in which addition of Fmoc protected amino acids to a resin of polystyrene is alternated with a Fmoc-deprotection procedure (see e.g., Gausepohl et al., 1990). Peptides are cleaved from the resin and side chain protective groups removed by treatment with aqueous TFA. Peptides are analyzed by reversed phase HPLC, lyophilized, dissolved at a concentration of 1 mg/ml in phosphate-buffered saline with 3% DMSO (Sigma, St. Louis, Mo.), and stored at −70° C.

### EXAMPLE 4

sMHC Assay for Deantigenized MAb T Cell Epitopes

To screen altered MAb T cell epitopes for deantigenized epitopes, sMHC assays as described in Example 2 are used to screen altered T cell epitopes generated from Example 3. Altered MAb T cell epitopes exhibiting a binding affinity less than that of the unaltered epitope (as determined in Example 2) identify deantigenized T cell epitopes according to this invention.

### EXAMPLE 5

Generation of a Modified MAb

To engineer a modified MAb, exhibiting reduced immunogenicity compared to that of the unmodified MAb, the unaltered T cell epitope sequence found in the MAb is replaced with the deantigenized MAb T cell epitope sequence determined from Example 4, using genetic engineering techniques well known in the art.

Briefly, the modified MAb is engineered by spliced overlap extension polymerase chain reaction (SOEPCR; Eorton et al., 1989) using the expression vector encoding the unaltered MAb as the template. For each modified gene, the amplified product is purified and ligated into the MAb expression vector. Genetic constructs are confirmed by sequencing the modified MAb coding region.

### EXAMPLE 6

Production of a Modified MAb

Modified MAb is expressed and purified from transformed CHO cells following techniques well known in the art (Kaymakalan et al., 2000). Briefly, a recombinant expression vector encoding the modified MAb heavy and light chains is introduced into dhfr-CHO cells (Urlaub, G. and Chasin, L. A. (1980) Proc. Natl. Acad. Sci. USA 77:4216-4220) by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification.

### EXAMPLE 7

Reduced Immunogenicity of a Modified MAb

To examine reduced immunogenicity of the modified MAb, the modified MAb is computer modeled as described earlier, and the interaction energy with MHC compared to that of the unaltered anti-IL-12 antibody.

Additionally, the modified MAb sequence is subjected to “threading” through the MHC binding groove, as described earlier, to determine if any new T cell epitopes are generated as a result of the modification.

Finally, cellular immune response to the modified MAb is compared to that of the unaltered MAb. Cell-based assays are employed following the disclosure of Wamerdam et al., 2001, which is incorporated herein by reference. In some instances, the immunogenicity of the parent MAb and the modified MAb is compared in vivo in animals such as primates and HLA-transgenic mice.

### Conclusion

The totality of these analyses demonstrate that the immunogenicity of a heterologous protein can be reduced by the replacement of T cell epitopes with deantigenized T cell epitopes, which deantigenized epitopes are identified based upon reduced binding to sMHC (class I or II) molecules. As a consequence of reducing or eliminating epitope binding to MHC molecules, T-cell activation will be reduced or eliminated, therefore reducing the overall immunogenicity of any modified protein wherein the original T cell epitope is replaced with the deantigenized T cell epitope.

### References

Adair et al., 1991, WO 91/09967.

Altuvia et al., 1995, J. Mol. Biol. 249:244-250.

Buus et al., 1987, Science 235:1352.

Carr, 1998, WO 98/52976.

Carr et al., 2000, WO 00/34317.

De Bruijn et al., 1991, Eur. J. Immunol. 21:2963-2970.

Delhaise et al., 1984, J Mol Graphics 2:103-106.

Devereux et al. 1984, NAR 12(1):387-395.

Estell and Harding, 1999, WO 99/53038.

Horton et al., 1989, Gene 77:61-68.

Falket al., 1991, Nature 351:290.

Fothergill et al., 1998, WO 98/59244.

Gausepohl et al., 1990, In: Proc. 11th American Peptide Symposium. (Rivier & Marshall, Ed.) ESCOM, Leiden. pp 1003-1004.

Hiemstra et al. 1997, PNAS USA 94(19):10313-10318.

Hunkapiller et al., 1983, Methods Enzymol. 91:399.

Hunt et al., 1992, Science 225:1261.

Kaymakalan et al., 2000, WO 00/56772.

Kaufman and Sharp, 1982. Mol. Biol. 159:601-621.

Kubo et al., 2000, U.S. Pat. No. 6,037,135.

Law et al., 1991, EP 0438310.

Pamer et al., 1991, Nature 353:852-955.

Queen, 1989, WO 89/09622.

Ritter et al., 2001, Cancer Res. 61(18):6851-6859.

Schaeffer et al., 1989, PNAS USA 86:4649.

Sette et al., 1989, PNAS USA 86:3296.

Sette et al., 1991, In: Seminars in Immunology, V.3 (Gefter, ed) W. B. Saunders, Philadelphia, pp 195-202.

Stickler et al., 2000. J. Immunotherapy 23(6):654-660.

Taneja and David, 1998. J Clin Invest. 101(5):921-926.

Vitiello et al., 2001. U.S. Pat. No. 6,322,789

Urlaub and Chasin, 1980. PNAS USA 77:4216-4220.

Warmerdam et al., 2001, WO 01/40281.

Winter, 1989, EP 0239400.

All of the publications cited herein are hereby incorporated by reference in their entirety.

I/we claim:

1. A method for detecting a deantigenized T cell epitope, said method comprising:
(a) providing an amino acid sequence of a T cell epitope, said T cell epitope having a binding affinity to a soluble MHC molecule;
(b) providing one or more altered T cell epitopes, wherein the amino acid sequence of said altered T cell epitope is different from the amino acid sequence of said T cell epitope;
(c) contacting said altered T cell epitope with said soluble MHC molecule for sufficient time to permit MHC-epitope binding complexes to form; and
(d) detecting one or more altered T cell epitopes, wherein said detected altered T cell epitope identifies a deantigenized T cell epitope having a binding affinity to said soluble MHC molecule less than the binding affinity of said T cell epitope to said soluble MHC molecule.

2. The method of claim 1 further comprising the steps of:
(e) providing one or more altered T cell epitopes, wherein the amino acid sequence of said one or more altered T cell epitopes is different from the amino acid sequence of a ueantigenized T cell epitope obtained in step (d); and
(f) repeating steps (c) and (d).

3. The method of claim 1, wherein said deantigenized T cell epitope possesses a dissociation constant with said soluble MHC molecule greater than or equal to about 5×10⁻⁷ M.

4. The method of claim 1, wherein said deantigenized T cell epitope possesses a dissociation constant with said soluble MHC molecule greater than or equal to about 5×10⁻⁵ M.

5. The method of claim 1, wherein said deantigenized T cell epitope possesses a dissociation constant with said soluble MHC molecule greater than or equal to about 5×10⁻³ M.

6. A method for generating a modified polypeptide, wherein said modified polypeptide exhibits reduced immunogenicity compared to that of an immunogenic polypeptide, wherein the amino acid sequence of said immunogenic polypeptide comprises at least one T cell epitope amino acid sequence, said method comprising:
(a) detecting a deantigenized T cell epitope according to any one of the methods of claim 1; and
(b) generating a polypeptide having an amino acid sequence modified from said immunogenic polypeptide, such that said deantigenized T cell epitope amino acid sequence detected from step (a) is substituted for said T cell epitope amino acid sequence of said immunogenic polypeptide.

7. The method of claim 6, wherein said modified polypeptide exhibits a biological function similar to that exhibited by said immunogenic polypeptide.

8. A deantigenized T cell epitope detected by any one of the methods of claim 1.

9. A polynucleotide encoding a deantigenized T cell epitope according to claim 8.

10. An expression vector containing a polynucleotide according to claim 9.

11. A host cell transformed with a vector according to claim 10.

12. A modified polypeptide, wherein said modified polypeptide exhibits reduced immunogenicity compared to that of an immunogenic polypeptide, said modified polypeptide generated by any one of the methods of claim 1.

13. The modified polypeptide according to claim 12, wherein said modified polypeptide is selected from the group of modified polypeptides that: exhibit enzymatic activity; act as an adjuvant; function as a carrier for other molecules; and are capable of binding to a molecule within or administered to an animal to alter the bioactivity, biodistribution and/or bioavailability of the bound molecule.

14. The modified polypeptide according to claim 12, wherein said modified polypeptide is a modified immunoglobulin.

15. The modified polypeptide according to claim 12, wherein said modified polypeptide is a modified monoclonal antibody.

16. A pharmaceutical composition comprising a modified polypeptide according to any one of the modified polypeptides of claim 12 and pharmaceutically acceptable carrier.

17. Use of a modified polypeptide according to any one of the modified polypeptides of claim 12 to prevent, to treat, or to diagnose a disease or disorder in a vertebrate.

18. A polynucleotide encoding a modified polypeptide according to any of claim 12.

19. An expression vector containing a polynucleotide according to claim 18.

20. A host cell transformed with a vector according to claim 19.
